(19) United States
(12) Patent Application Publication
(10) Pub. No.: US 2013/0041382 A1
(43) Pub. Date: Feb. 14, 2013

(54) Injector Apparatus for Injecting Intraocular Lens

(71) Applicant: Joshua Ben Nun, Bet Herut (IL)

(72) Inventor: Joshua Ben Nun, Bet Herut (IL)

(73) Assignee: Nulens Ltd., Herzliya Pituah (IL)

(21) Appl. No.: 13/643,010

(22) PCT Filed: May 8, 2011
(86) PCT No.: PCT/IL2011/000366

§ 371 Date: Oct. 23, 2012

(30) Foreign Application Priority Data
May 6, 2010 (IL) 205585
Oct. 11, 2010 (IL) 208621

(51) Int. Cl. A61F 9/007

(52) U.S. Cl. 606/107

(57) ABSTRACT

Injector apparatus (100a, 100b) including a syringe-like injector (200a, 200b), an intraocular lens support arrangement (207, 301) for initially supporting an intraocular lens (10) and a pair of compression members (208a, 208b, 302a, 302b). The syringe-like injector includes an elongated injector base (202) having a trailing handheld end (203, 3070) and a leading delivery end (204), and a hand operated injector plunger (207, 276) slidingly mountable on the injector base for manual urging from an initial outward set-up position to a final inward injection position for injecting the intraocular lens. The compression members compress the intraocular lens at the leading delivery end prior to manually urging the injector plunger to its final inward injection position for injecting the intraocular lens.

### FIELD OF THE INVENTION

The invention relates to injector apparatus for injecting intraocular lenses.

### BACKGROUND OF THE INVENTION

Intraocular lens implantations into human eyes involve making incisions. Incisions are preferably as small as possible to reduce trauma and speed healing. Some intraocular lenses are required to be implanted in a particular orientation to function correctly.

Exemplary injector apparatus for injecting intraocular lenses is illustrated and described in inter alia U.S. Pat. No. 5,643,276 to Zaleski, U.S. Pat. No. 5,947,974 to Brady et al., U.S. Pat. No. 5,947,976 to Van Noy et al., U.S. Pat. No. 5,976,150 to Copeland, U.S. Pat. No. 6,010,510 to Brown et al., U.S. Pat. No. 6,251,114 to Farmer et al., U.S. Pat. No. 6,723,104 to Ott, U.S. Pat. No. 7,279,006 to Vincent, US Patent Application Publication No. 2004/0111094 to Meyer, US Patent Application Publication No. 2005/0033308 to Callahan et al., US Patent Application Publication No. 2005/0149056 to Rathert, US Patent Application Publication No. 2005/0149057 to Rathert, US Patent Application Publication No. 2005/0222577 to US Patent Application Publication No. 2005/0222579 to Vaquero et al., US Patent Application Publication No. 2005/0283164 to Wu et al., US Patent Application Publication No. 2006/0142781 to Pynson et al., US Patent Application Publication No. 2006/0229634 to Shepherd, US Patent Application Publication No. 2007/0050023 to Bessiere et al., US Patent Application Publication No. 2007/0150054 to Pynson, US Patent Application Publication No. 2007/0250068 to Vincent-Aubry, PCT International Publication No. WO2006/059183 to Pynson, PCT International Publication No. WO2006070219 to Pynson, and PCT International Publication No. WO2007/037689 to Wanders.

### SUMMARY OF THE INVENTION

The present invention is directed towards injector apparatus for injecting an intraocular lens in a human eye. The injector apparatus is intended for use with intraocular lenses having an optical axis and including an optical lens and at least two outward extending haptics for anchoring the intraocular lens in the human eye, the optical lens having a pair of opposite optical surfaces and being compressible on application of opposite compression forces in a plane perpendicular to its optical axis from an unstressed generally circular shape to a stressed elongated shape whereupon the optical lens outwardly bulges along its optical axis with respect to its unstressed generally circular shape in accordance with commonly owned PCT International Application No. PCT/IL2008/000284 published under PCT International Publication No. WO 2008/107882, the contents of which are incorporated herein by reference. The intraocular lens may be intended for implantation either as an independent intraocular item or in combination with a discrete base member in accordance with aforesaid PCT International Application No. PCT/IL2008/000284. The former can have a fixed Diopter strength or a continuously variable Diopter strength in accordance with U.S. Pat. No. 7,220,279 to Ben Nun, the contents of which are incorporated herein by reference. The latter has a continuously variable Diopter strength in accordance with aforesaid PCT International Application No. PCT/IL2008/000284.

The injector apparatus of the present invention includes (a) a syringe-like injector having an elongated injector base having a trailing handheld end and a leading delivery end, and a hand operated injector plunger slidingly mounted on the injector base for being manually urged from an initial outward set-up position to a final inward injection position for injecting the intraocular lens, (b) an intraocular lens support arrangement for initially supporting the intraocular lens in its unstressed state such that its optical lens overlies the injector base with a topside optical surface facing away from the injector base and an underside optical surface facing toward the injector base and (c) a pair of compression members for applying the opposite compression forces to the intraocular lens for compressing its optical lens into its stressed elongated shape at the leading delivery end prior to the injector plunger being manually urged to the final inward injection position for injecting the intraocular lens into the human eye.

The intraocular lens support arrangements preferably initially support intraocular lenses such that their underside optical surfaces do not contact an underlying surface. The intraocular lens support arrangements typically employ at least one of the compression members as a support surface for initially supporting an intraocular lens. The opposite compression forces are the result of relative movement of the pair of compression members towards one another. Such relative movement can be the result of a sliding action. Alternatively, such relative movement can be the result of an inward pivot action. Either both of the compression members may undergo a movement or possibly one compression member may move with respect to a stationary compression member. The pair of compression members preferably apply the opposite compression forces at the leading delivery end to avoid forward displacement of the intraocular lens in its stressed elongated shape except for the final launching of an intraocular lens into a human eye for implantation purposes. Intraocular lens support arrangements preferably include a lens support for supporting an underside optical surface facing towards an injector base from bulging downwards on application of opposite compression forces to avoid same being scratched or otherwise defected during the injection process such that the compression causes the upwards bulging of the topside optical surface only.

The injector apparatus can be designed such that different series of user actions are required to apply the opposite compression forces to squeeze the intraocular lens to its stressed elongated shape prior to forward displacement of the injector plunger to its final inward injection position for injecting the intraocular lens into a human eye. The injector apparatus preferably includes a first safety latch arrangement for preventing inadvertent compression of an intraocular lens and a second safety latch arrangement for preventing inadvertent manual urging of the injector plunger to its final inward injection position. The safety latch arrangements require specific user actions to release apparatus components. Such user actions can include the removal of a safety latch member, rotation of a safety latch member from a blocking position to an unblocking position, sliding of a safety latch member from a blocking position to an unblocking position, and the like.

The injector apparatus can be designed with a syringe-like injector having a pre-loaded intraocular lens thereby precluding the need for loading an intraocular lens. Alternatively, the injector apparatus can be designed with a syringe-like injector for use with interchangeable cartridges each storing an intraocular lens thereby reducing inventory for storing a range of intraocular lenses of different sizes, optical power, and the like. The interchangeable cartridges may or may not include the pair of compression members. The injector apparatus can be equally used by left and right hand users.

### BRIEF DESCRIPTION OF DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a perspective view of an intraocular lens including an annular haptics main body with a pair of outwardly extending haptics and an optical lens;

FIG. 2 is a left elevation view of FIG. 1's intraocular lens;

FIG. 3 is a perspective view of the intraocular lens being compressed by opposite compression forces into a stressed elongated shape for implantation through a small incision into a human eye;

FIG. 4 is a perspective view of injector apparatus with a pre-loaded intraocular lens in accordance with a first preferred embodiment of the present invention;

FIG. 5 is a partially exploded perspective view of FIG. 4's injector apparatus;

FIG. 6 is an exploded perspective view of FIG. 4's injector apparatus;

FIG. 7A is a top view of FIG. 4's injector base;

FIG. 7B is a transverse cross section of FIG. 4's injector base along line A-A in FIG. 7A;

FIG. 8 is a perspective view of the left and right compression members of FIG. 4's injector apparatus;

FIG. 9 is a perspective view of the injector plunger of FIG. 4's injector apparatus;

FIG. 10 is a perspective view of the base member of FIG. 4's injector apparatus;

FIG. 11A is a perspective view of the injector apparatus in its set-up position;

FIG. 11B is a perspective view of the injector apparatus subsequent to removal of a cartridge locking member;

FIG. 11C is a perspective view subsequent to user manually urging the injector plunger through an intermediate position for urging a lens support upwards to support an underside optical surface of the intraocular lens;

FIG. 11D is a longitudinal cross section of FIG. 11C's injector apparatus along line B-B in FIG. 11C;

FIG. 11E is a perspective view subsequent to the user manually urging the injector plunger until the compression members stop at the end of their compression member tracks at the leading delivery end;

FIG. 11F is a perspective view subsequent to partial insertion of the intraocular lens through a pre-formed corneal incision into a subject's eye and manually urging the injector plunger to its final inward injection position for fully injecting the intraocular lens in the subject's eye;

FIG. 12 is a dissembled perspective view of injector apparatus including an interchangeable cartridge in accordance with a second preferred embodiment of the present invention;

FIG. 13 is a perspective view of FIG. 12's syringe-like injector;

FIG. 14 is a longitudinal cross section of FIG. 12's syringe-like injector along line C-C in FIG. 13;

FIG. 15 is an exploded view of FIG. 12's syringe-like injector;

FIG. 16 is a partially dissembled perspective view of FIG. 12's cartridge with a pre-installed IOL and a detached cartridge locking member;

FIG. 17 is a longitudinal cross section view of the FIG. 12's cartridge along line D-D in FIG. 12;

FIG. 18 is an exploded view of FIG. 12's cartridge;

FIG. 19 is a close-up perspective view of a driven plunger of FIG. 12's cartridge;

FIG. 20 is a close-up side view of FIG. 19's driven plunger;

FIG. 21A is a perspective view of FIG. 12's injector assembly in a dissembled state;

FIG. 21B is a perspective view of an assembled injector assembly subsequent to a forward displacement of its cartridge prior to its being stopped at an intermediate operative position;

FIG. 21C is a perspective view of the injector assembly subsequent to the user manually urging the injector plunger forward to snap fit with the driven plunger and upward removal of the driven plunger stopper;

FIG. 21D is a perspective view of the injector apparatus in the same operative state as in FIG. 21C with the cartridge locking member shown transparent to show the IOL in its initial non-stressed state in the cartridge;

FIG. 21E is a perspective view of the injector apparatus with the cartridge locking member shown transparent subsequent to the user manually urging the injector plunger forward to urge the intraocular lens to the leading edges of the compression members ready for compression;

FIG. 21F is a longitudinal cross section of the injector assembly in the same operative state as in FIG. 21E along line E-E in FIG. 21E;

FIG. 21G is a perspective view showing the upward detachment of the cartridge locking member to release the compression members enabling their forward displacement for compression purposes;

FIG. 21H is a perspective view of the injector assembly showing the user pushing the cartridge base forward to compress the intraocular lens into a stressed elongated shape; and

FIG. 21I is a perspective view of the injector assembly subsequent to to partial insertion of the intraocular lens through a pre-formed corneal incision into a subject's eye and manually urging the injector plunger to its final inward injection position for fully injecting the intraocular lens in the subject's eye.

### DETAILED DESCRIPTION OF PREFERRED EMBODIMENTS OF THE INVENTION

### Description of Intraocular Lens (IOL)

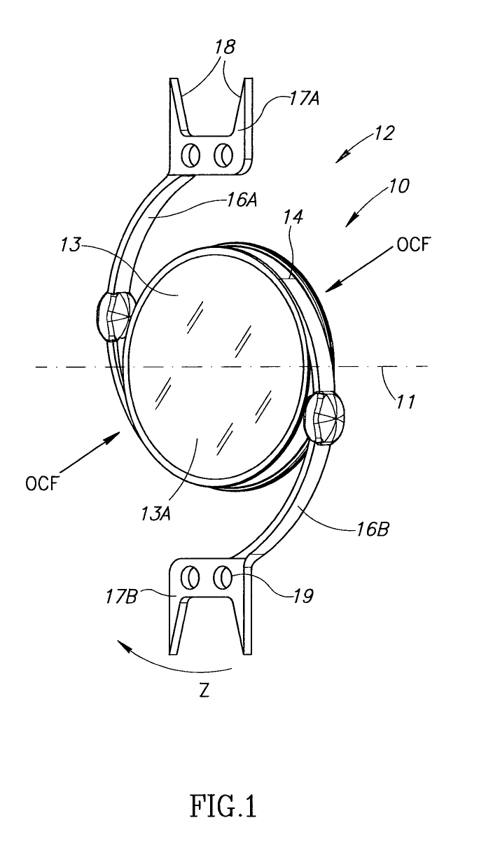

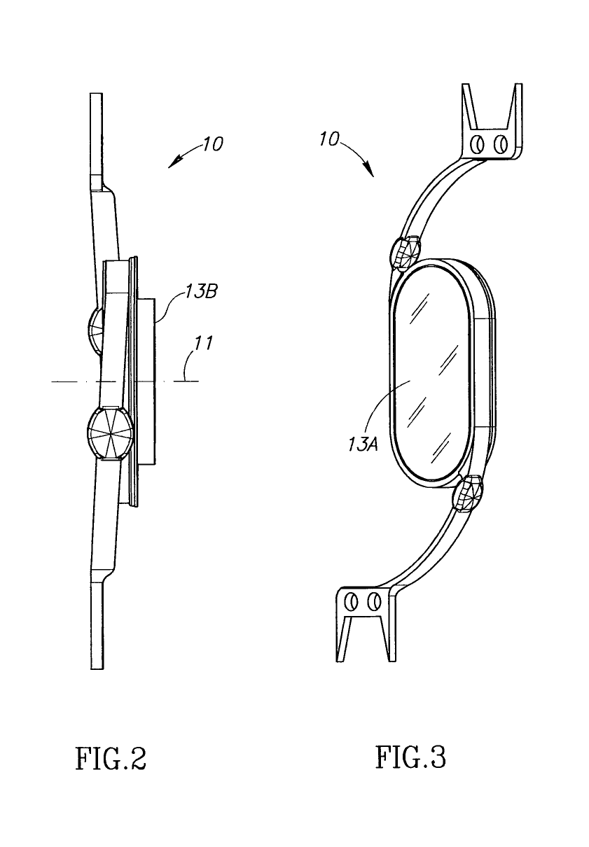

FIGS. 1 to 3 show an intraocular lens (IOL) 10 have an optical axis 11 intended to be co-directional with a human eye's visual axis on implantation in a human eye. The IOL 10 includes a haptics system 12 for positioning the IOL in a predetermined position in a human eye and an optical lens 13 including a leading optical surface 13A and a trailing optical surface 13B. The haptics system 12 is made from suitable rigid bio-compatible transparent polymer material such as PMMA, and the like. The optical lens 13 is made from suitable bio-compatible transparent material. The IOLs 10 are pre-assembled using conventional assembly techniques, for example, gluing, soldering, and the like. The haptics system 12 includes an annular haptics main body 14 with diametrically opposite elongated generally C-shaped left and right haptics 16A and 16B extending in opposite directions in a plane perpendicular to the optical axis 11. The haptics main body 14 contains the leading optical surface 13A and the trailing optical surface 13B is posterior thereto. The haptics 16 are capable of being flexed around the haptics main body 14 as shown by the arrow labeled Z. The haptics 16A and 16B terminate at left and right bifurcated attachment plates 17A and 17B each having a pair of spaced apart puncturing members 18 and a pair of throughgoing manipulation bores 19. The haptics main body 14 is intended to be squeezable into an elongated shape on application of opposite compression forces denoted OCF for implantation through a small corneal incision into a human eye. The optical lens 13 outwardly bulges naturally bulges both forwards and backward along its optical axis 11 on application of the opposite compression forces OCF with respect to its unstressed generally circular shape.

Description of Injector Apparatus with Pre-loaded Intraocular Lens

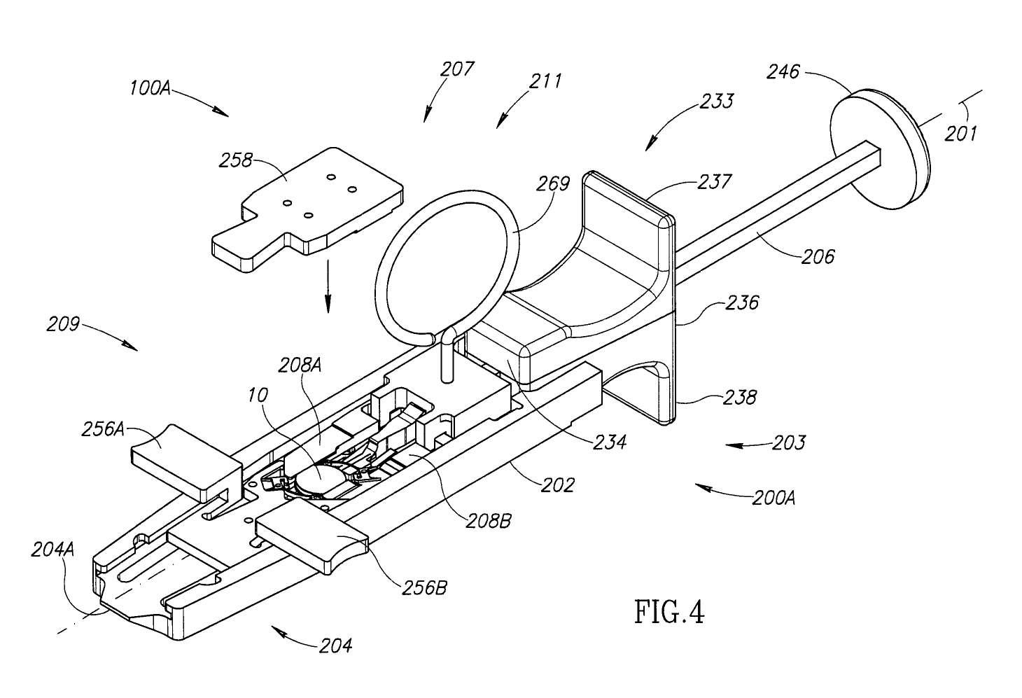

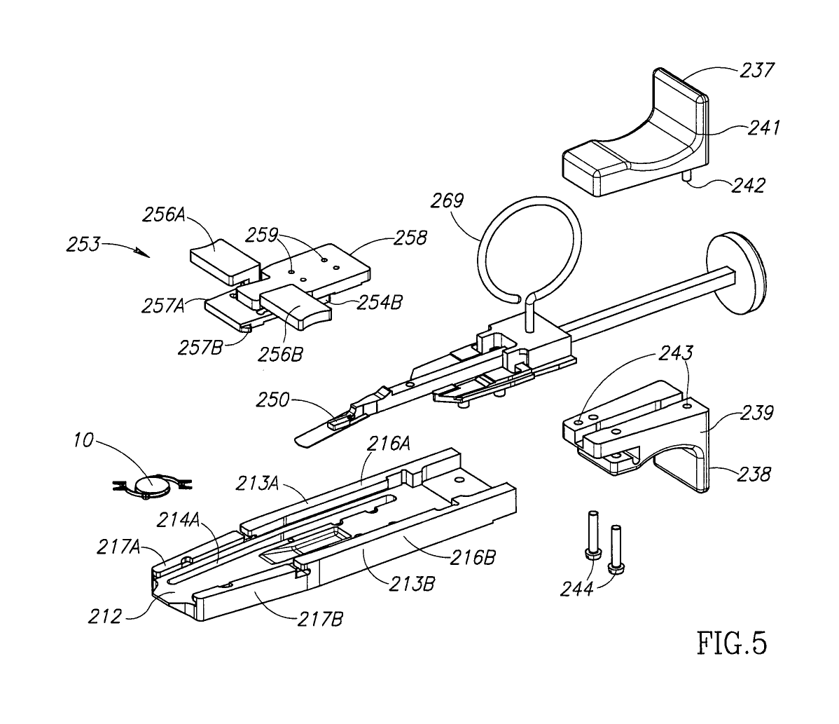

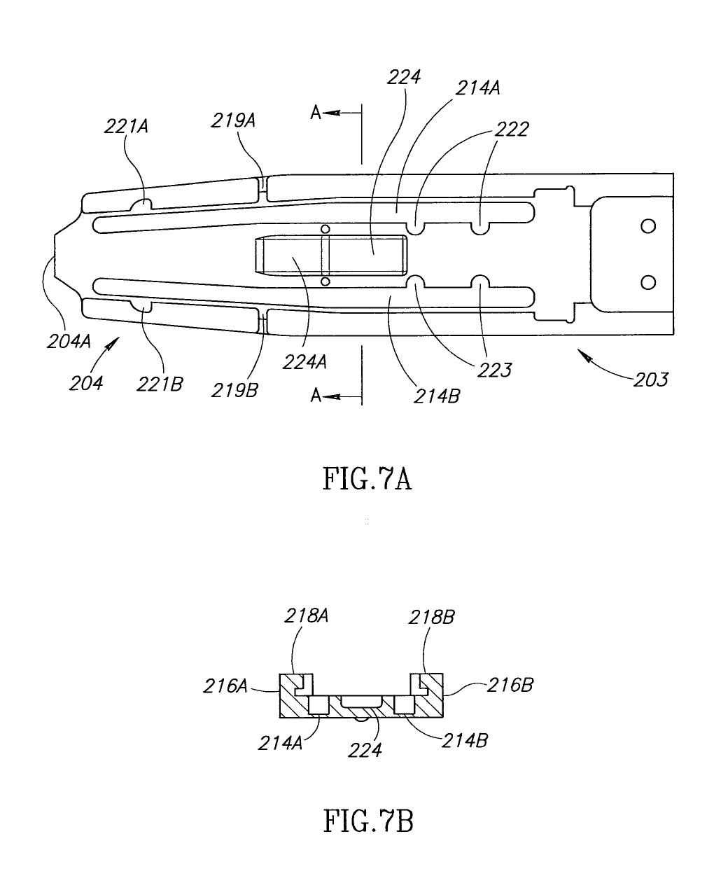

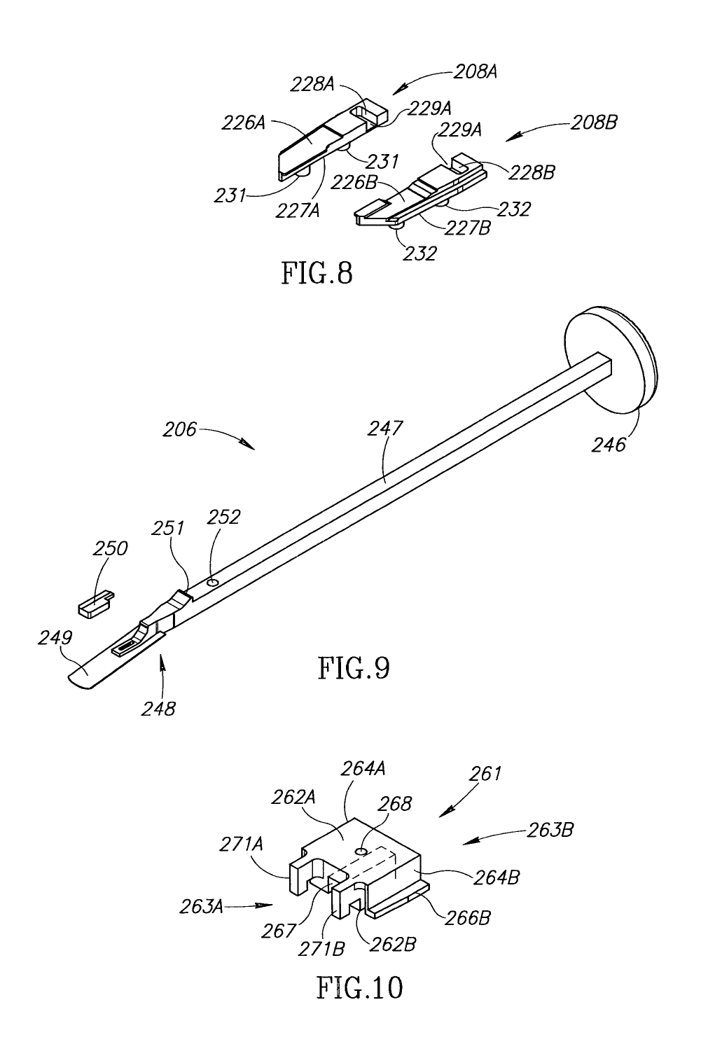

FIGS. 4 to 10 show injector apparatus 100A including a syringe-like injector 200A having a longitudinal axis 201 and an elongated injector base 202 having a trailing handheld end 203 and a leading delivery end 204 and an injector plunger 206 slidingly mounted on the injector base 202 for being manually urged from an initial outward set-up position to a final inward injection position for injecting a pre-loaded IOL 10, an intraocular lens support arrangement 207 for initially supporting the pre-loaded IOL 10 in a non-stressed state and a pair of longitudinal directed elongated compression members 208 for applying opposite compression forces OCF to the IOL 10 for compressing its optical lens 13 into its stressed elongated shape at the leading delivery end 204 prior to the injector plunger 206 being manually urged to its final inward injection position for injecting the IOL 10 into the human eye. The intraocular lens support arrangement 207 supports the IOL 10 overlying the injector base 202 with a topside optical surface 21A facing away from the injector base 202 and an underside optical surface 21B facing towards the injector base 202. The injector apparatus 100A includes a first safety latch arrangement 209 for preventing inadvertent compression of the IOL 10 and a second safety latch arrangement 211 for preventing inadvertent manual urging of the injector plunger 206 to its final inward injection position.

The injector base 202 includes an upper injector base surface 212 and opposite left and right side walls 213A and 213B. The upper injector base surface 212 includes opposite left and right longitudinal directed compression member tracks 214A and 214B for assisting in guiding the compression members 208 along the injector base 202. The side walls 213 include parallel trailing wall sections 216 from the trailing handheld end 203 to about midway therealong and converging leading wall sections 217 from midway to the leading delivery end 204 for inwardly urging the compression members 208 towards one another for applying the opposite compression forces to the IOL 10. The side walls 213 include side wall overhangs 218 for securing the compression members 208 in the injector base 202. The first safety latch arrangement 209 includes a pair of opposite sidewall cutouts 219 formed in the side walls 213 slightly forward of midway therealong and a pair of opposite overhang recesses 221 formed in the side wall overhangs 218 towards the leading delivery end 204.

The left compression member track 214A is formed with a pair of inward directed left indentations 222 for assisting in the insertion of the left compression member 208A in the injector base 202. The right compression member track 214B is formed with a pair of inward directed right indentations 223 for assisting in the loading of the right compression member 208B in the injector base 202. The leading delivery end 204 is formed with a protruding chute 204A slightly extending beyond the side walls 213. The upper injector base surface 212 is formed with a central longitudinal directed lens support recess 224 extending from midway therealong to the converging leading sections 217. The lens support recess 224 includes an upwardly inclined ramp 224A from midway along the injector base 202 to smoothly blend with the upper injector base surface 212 towards the leading delivery end 204.

The compression members 208A and 208B have upper surfaces 226A and 226B and lower surfaces 227A and 227B. The upper surfaces 226A and 226B are formed with opposite sideways directed trailing slots 228A and 228B having inward directed open ends 229A and 229B. The left lower surface 227A includes a pair of feet 231 for initial insertion into the left indentations 222 and subsequent sliding along left compression member track 214A. The right lower surface 227B is formed with a right pair of feet 232 for initial insertion into the right indentations 223 and subsequent sliding along the right compression member track 214B.

The trailing handheld end 203 is fitted with a handgrip 233 having a leading end 234 and a trailing end 236 with a pair of oppositely directed upward and downward finger supports 237 and 238. The handgrip 233 has a dual component construction including a base handgrip component 239 and an upper handgrip component 241 for mounting on the base handgrip component 239. The base handgrip component 239 includes a longitudinal directed plunger channel 239A for sliding passage of the injector plunger 206 therealong. The upper handgrip component 241 includes positioning pins 242 (not shown) for insertion into positioning holes 243 formed in the base handgrip component 239 for assisting in the assembly of the handgrip 233. The handgrip 233 includes assembly screws 244 for assembling the base handgrip component 239 and the upper handgrip component 241 on the trailing handheld end 203. Alternative assembly techniques can be employed for assembling the handgrip 233 including snap fit, gluing, and the like.

The injector plunger 206 includes a thumb depressed plunger head 246 and a shaft 247 having a leading shaft end 248 opposite the plunger head 246. The leading shaft end 248 includes a lens support 249 forming part of the intraocular lens support arrangement 207 which is initially disposed in the lens support recess 224 beneath the IOL 10 and urged upwards along the ramp 224A to support the underside optical surface 21B on forward displacement of the injector plunger 206. The leading shaft end 248 is also provided with a silicon cushion 250 for pushing against the trailing portion of the haptics main body 14 during forward displacement of the injector plunger 206. The silicon cushion 250 is intended to be compressed due to elongation of the IOL 10 on application of the opposite compression forces to minimize relative displacement of the IOL 10 to the compression members 208 during compression of the IOL 10. The leading shaft end 248 is further provided with a plunger stop 251 trailing the lens support 249 for preventing the injector plunger 206 from inadvertently sliding out from the rear of the injector apparatus 100A on backwards tilting of the same and a plunger blind bore 252 of the safety latch arrangement 211.

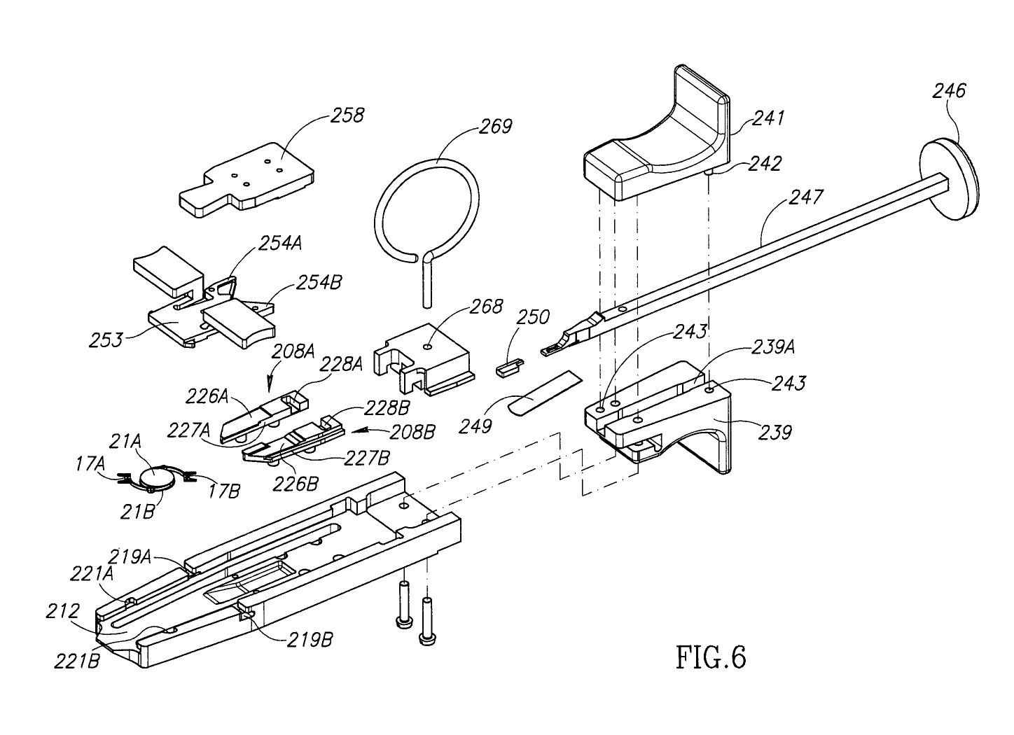

The safety latch arrangement 209 includes a cartridge locking member 253 initially disposed between the leading delivery end 204 and the compression members 208. The cartridge locking member 253 includes a pair of resiliently mounted wings 254 which extend under the side wall overhangs 218 and which are formed with finger operated release members 256 outwardly extending through the sidewall cutouts 219. The cartridge locking member 253 has a pair of leading outwardly protruding protrusions 257 for alignment with the overhang recesses 221 on forward displacement of the cartridge locking member 253 for enabling its upward removable from the injector 200A. The cartridge locking member 253 is formed with a trailing transparent cover 258 for covering the underlying IOL 10 and enabling viewing of same. The transparent cover 258 is formed with throughgoing bores 259 for sterilization purposes. The cartridge locking member 253 is also provided with a support (not shown) for supporting the left attachment plate 17A in the set-up position of the injector apparatus 100A. FIGS. 4 and 6 show the cover 258 detached from the cartridge locking member 253 for illustrative purposes.

The safety latch arrangement 211 includes a generally square base member 261 disposed between the compression members 208 and the handgrip 233. The base member 261 includes opposite upper and lower major surfaces 262A and 262B, opposite leading and trailing surfaces 263A and 263B and opposite left and right minor surfaces 264A and 264B. The left and right minor surfaces 264A and 264B are formed with left and right flanges 266A and 266B for extending under the side wall overhangs 218A and 218B. The lower surface 262B is formed with a central longitudinal directed base member channel 267 for passage of the injector plunger 206 therethrough. The upper surface 262A is formed with a throughgoing bore 268 for registration with the plunger blind bore 252 in the injector plunger 206's initial outward set-up position. The safety latch arrangement 211 includes a manually removable safety pin 269 for initial insertion through the throughgoing bore 268 into the plunger blind bore 252 for engaging the injector plunger 206 with the base member 261. The leading surface 263A is formed with a pair of forward directed guides 271 for engaging the slots 228. The plunger stop 251 stops against the leading surface 263A for preventing the injector plunger 206 from inadvertently sliding out from the injector apparatus 100A on backwards tilting of same.

The initial set-up position of the injector apparatus 100A is as follows: The injector plunger 206 is in its initial outward set-up position. The intraocular lens support arrangement 207 supports the IOL 10 in its unstressed state in a diagonal position across the injector 200A with its left attachment plate 17A supported by the cartridge locking member 253 forward of the left compression member 208A, its right attachment plate 17B resting on the right compression member 208B's upper surface 226B, its topside optical surface 21A facing away from the injector base 202 and its underside optical surface 21B facing towards the injector base 202. The compression members 208 gently press against opposite sides of the IOL 10 but without compressing same. The compression members 208 are at their widest positions relative to the guides 271. The safety pin 269 is inserted through the base member throughgoing bore 268 into the plunger blind bore 252. The cartridge locking member 253 is securely mounted on the injector 200A.

Operation of Injector Apparatus with Pre-loaded Intraocular Lens

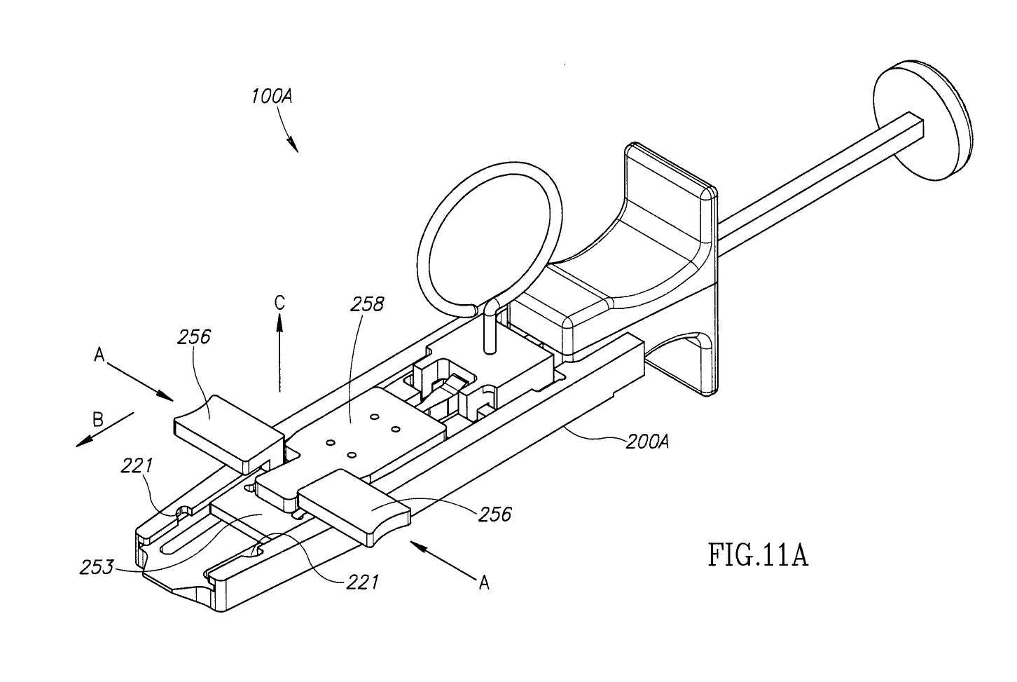

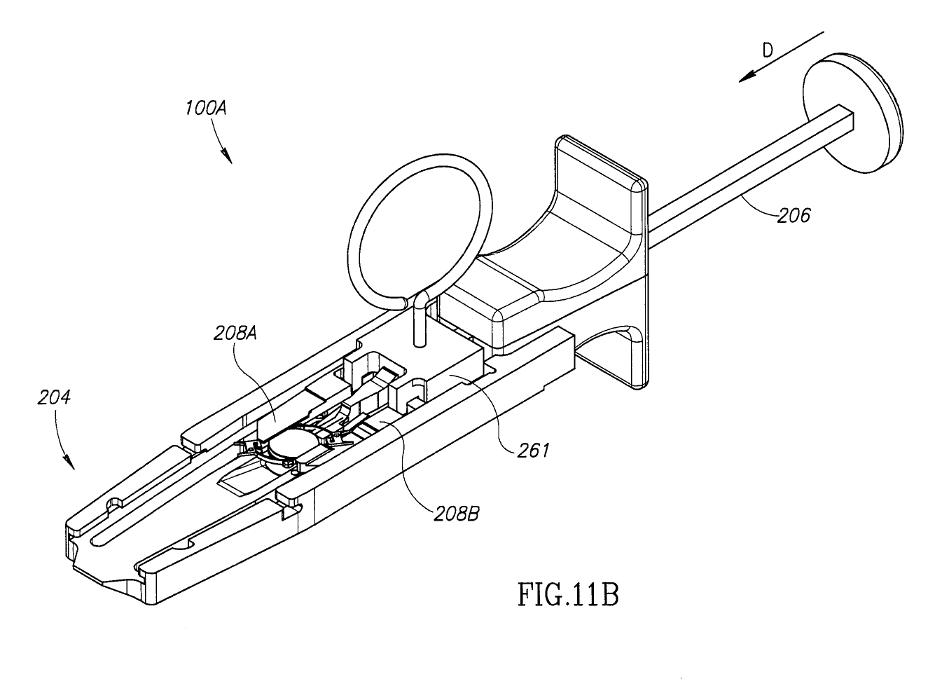

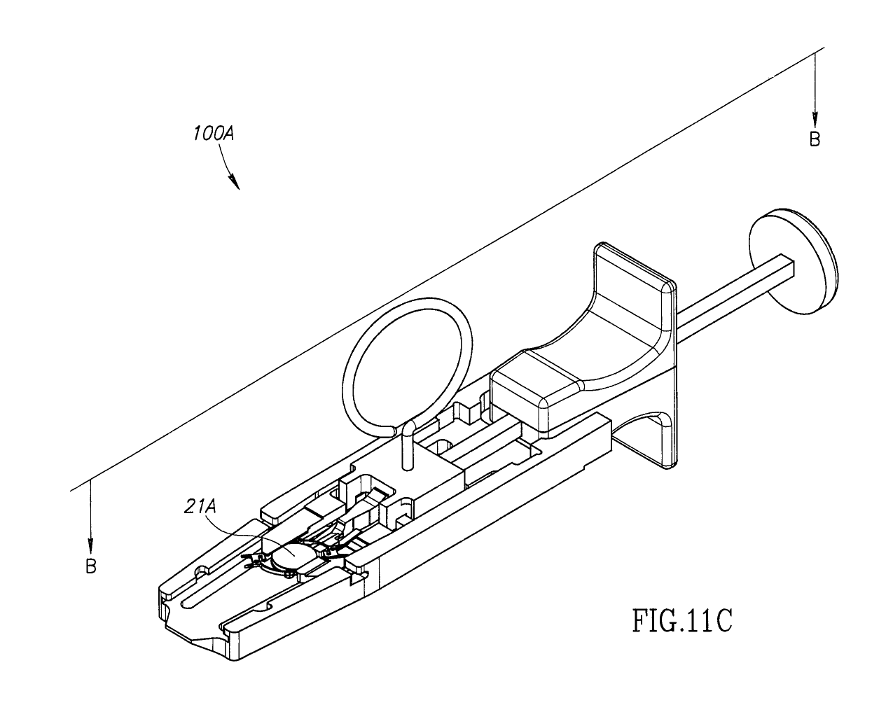

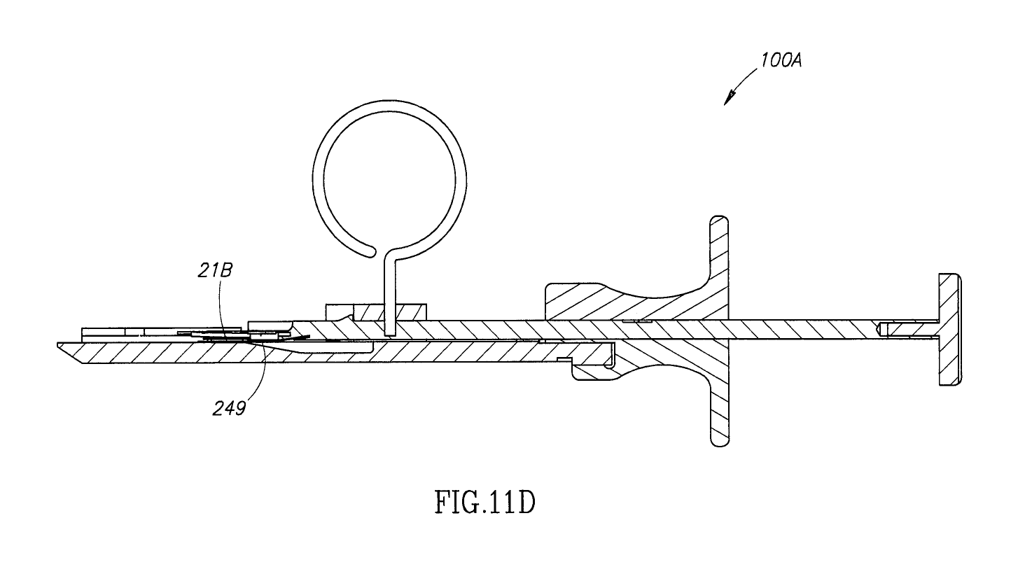

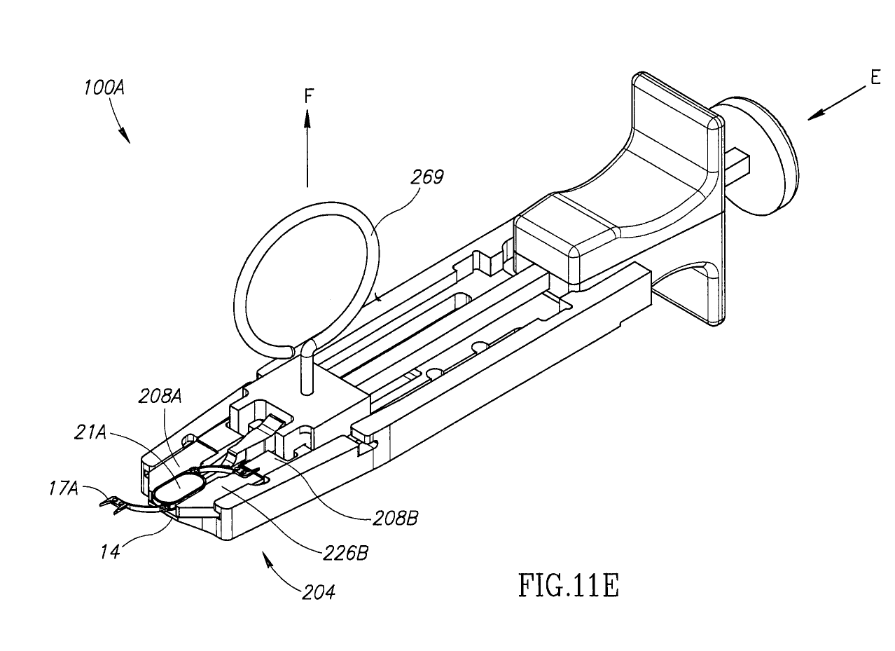

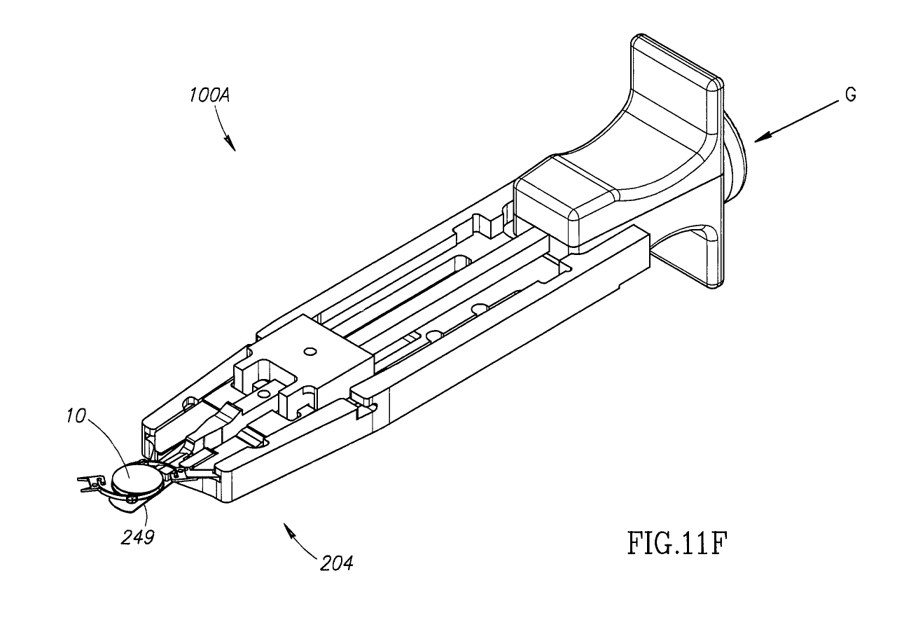

FIGS. 11A to 11F show operation of injector apparatus 100A as follows:

A user presses inwards on the finger operated release members 256 denoted by arrow A for inwardly pushing the resiliently mounted wings 254, pushes the cartridge locking member 253 as denoted by arrow 13 until the leading locking protrusions 257 are aligned with the overhang recesses 221 and removes the cartridge locking member 253 together with the transparent cover 258 from the injector 200A as denoted by arrow C (see FIGS. 11A and 11B).

The user manually urges the injector plunger 206 towards the leading delivery end 204 as denoted by arrow D which in turn urges the base member 261 and the compression members 208 forward toward the leading delivery end 204 (see FIGS. 11B and 11C). The injector plunger 206 passes through an immediate position for upward urging of the lens support 249 to support the underside optical surface 21B (see FIG. 11D).

The user continues to manually urge the injector plunger 206 as denoted by arrow E until the compression members 208 stop at the end of their compression member tracks 214 at the leading delivery end 204 (see FIG. 11E). The compression members 208 progressively apply the opposite compression forces to the IOL 10 during their forward displacement along the converging leading sections 217 such that the IOL 10 is in its stressed elongated shape at the leading delivery end 204. The opposite compression forces applied to the IOL 10 causes its topside optical surface 21A to bulge upwards whilst the lens support 249 prevents the underside optical surface 21B from bulging downwards. The elongation of the IOL 10 causes the left attachment plate 17A to extend further forward beyond the left compression member 208A and suspended from the haptics main body 14. The elongation of the IOL 10 causes the right attachment plate 17B to slightly extend rearward on the right compression member's upper surface 226B.

The user removes the safety pin 269 as denoted by arrow F to release the injector plunger 206 from the base member 261 (see FIG. 11E). The user inserts the leading left attachment plate 17A and the chute tip 204A through a corneal incision prepared in a subject's cornea and then presses the injector base 202 against the subject's cornea for partial insertion of the IOL 10 into the subject's eye. For the final step, the user manually urges the injector plunger 206 as denoted by arrow G to its final inward injection position for manually urging the lens support 249 beyond the leading delivery end 204 for fully injecting the IOL 10 into the subject's eye (see FIG. 11F). The IOL 10 reverts to its original non-stressed shape within the subject's eye ready for implantation. The user withdraws the injector apparatus 100A and proceeds to implant the IOL 10 as illustrated and described in commonly owned PCT International Application No. PCT/IL2007/001056 entitled Intraocular Lens Implantation Kit and published under PCT International Publication No. WO 2008/023379.

Description of Injector Apparatus with Interchangeable Cartridge

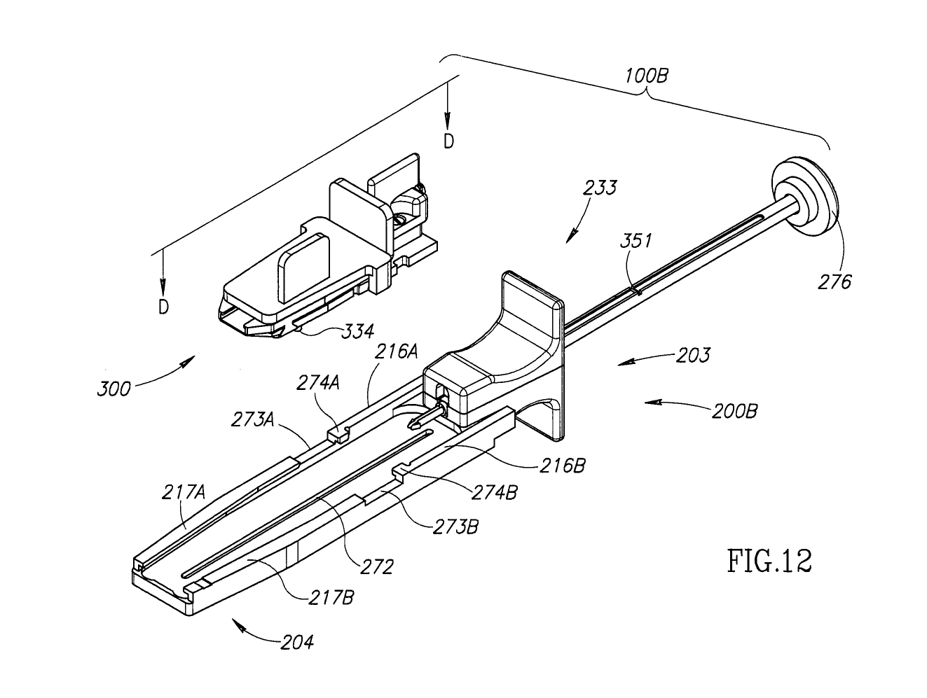

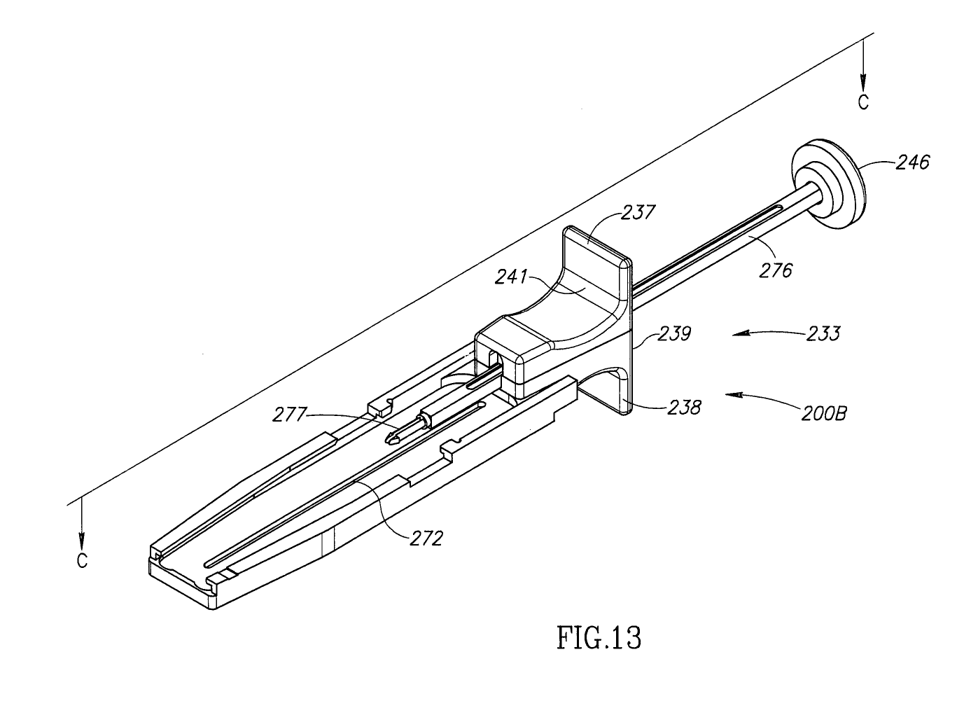

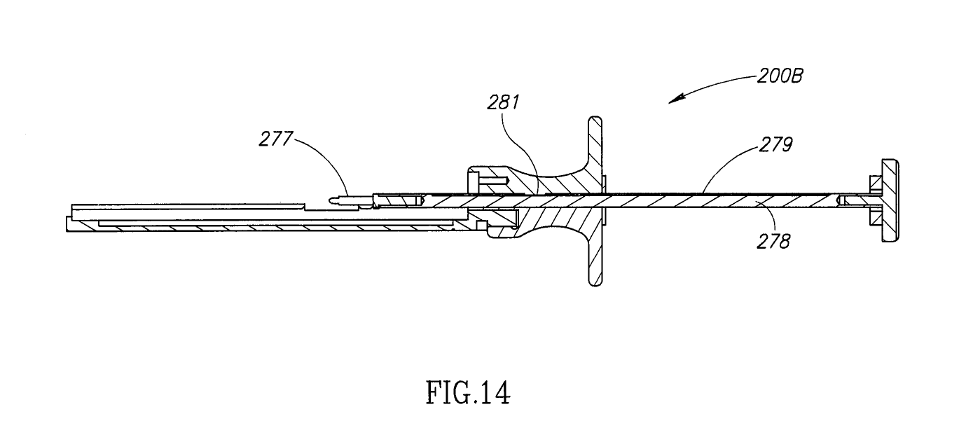

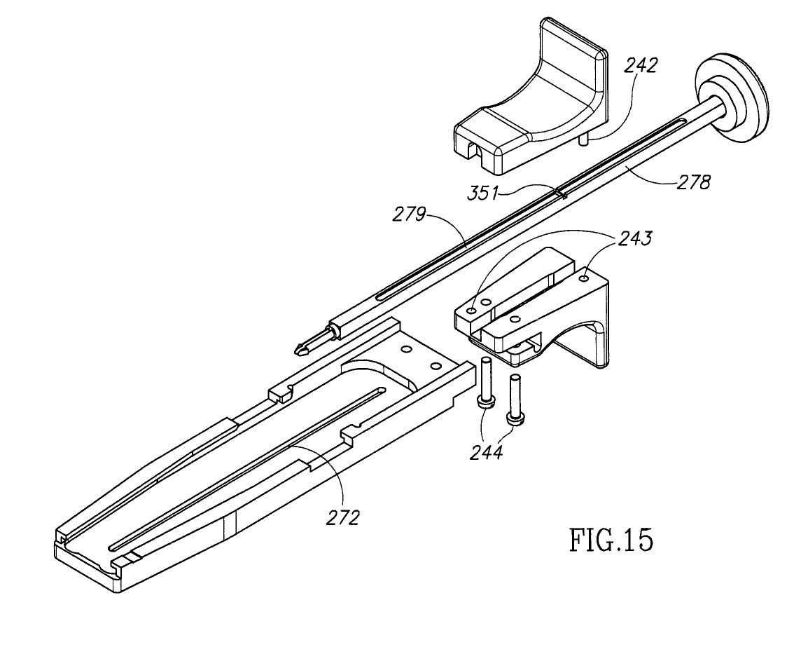

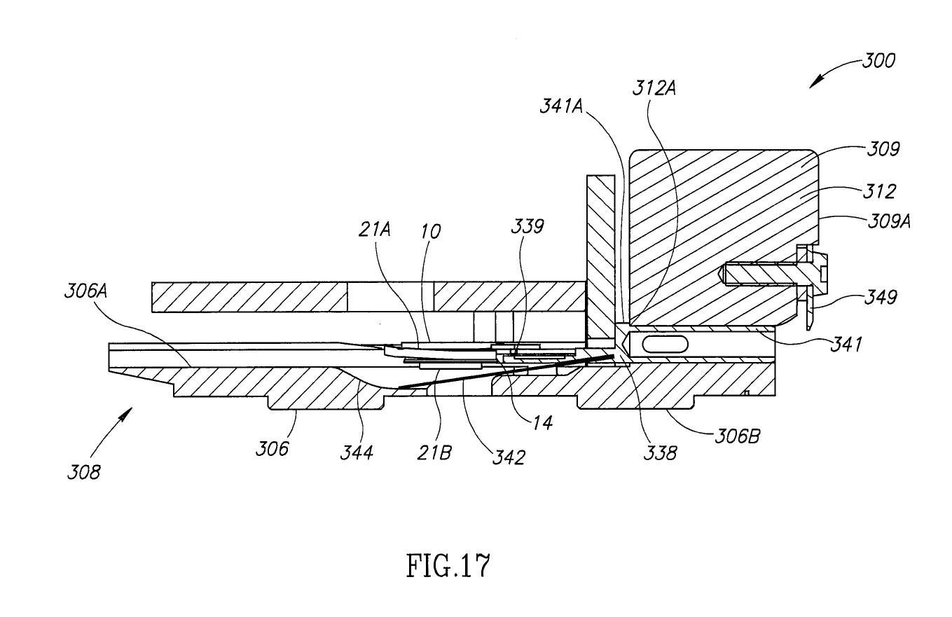

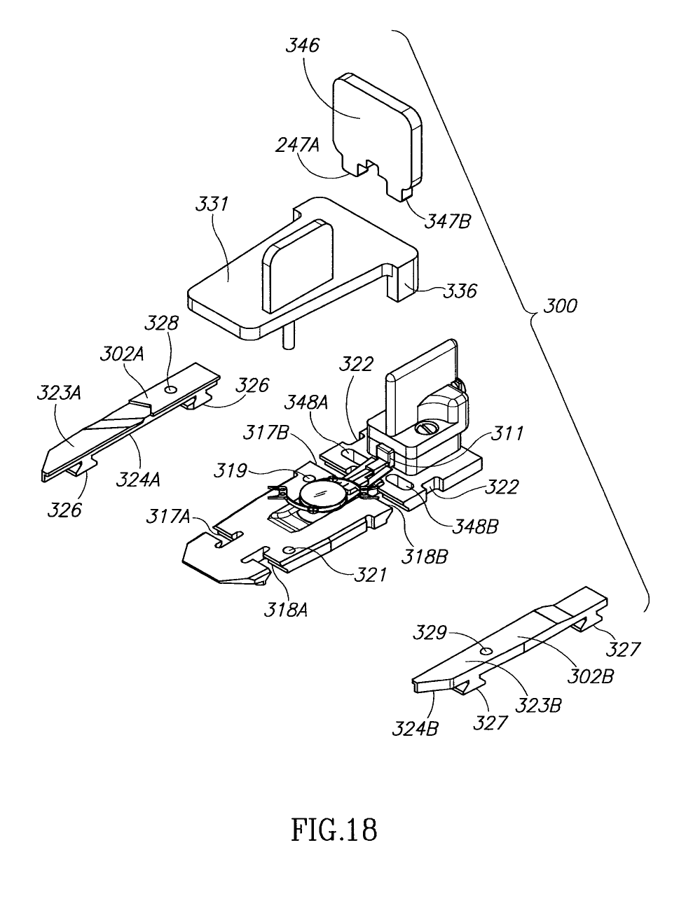

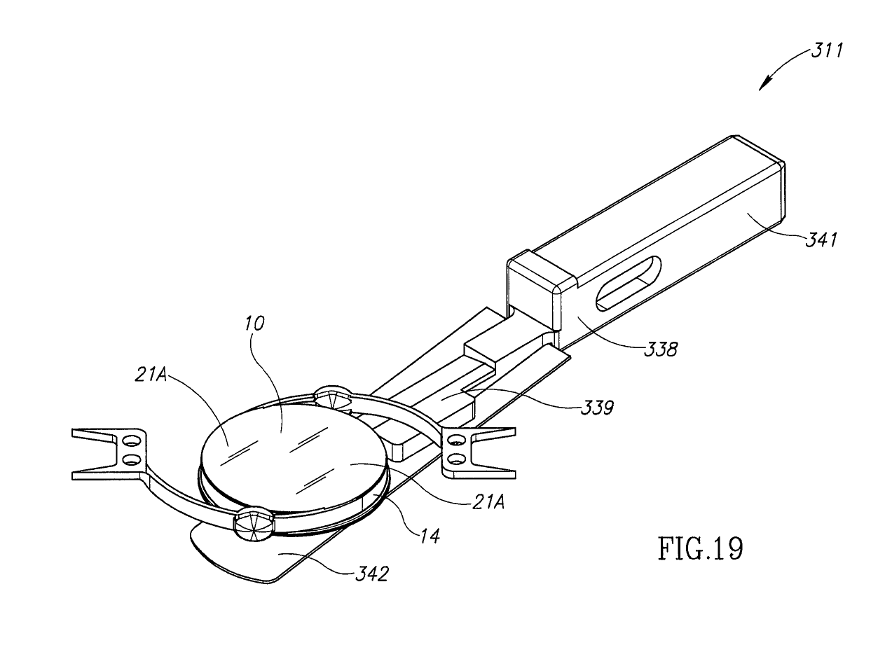

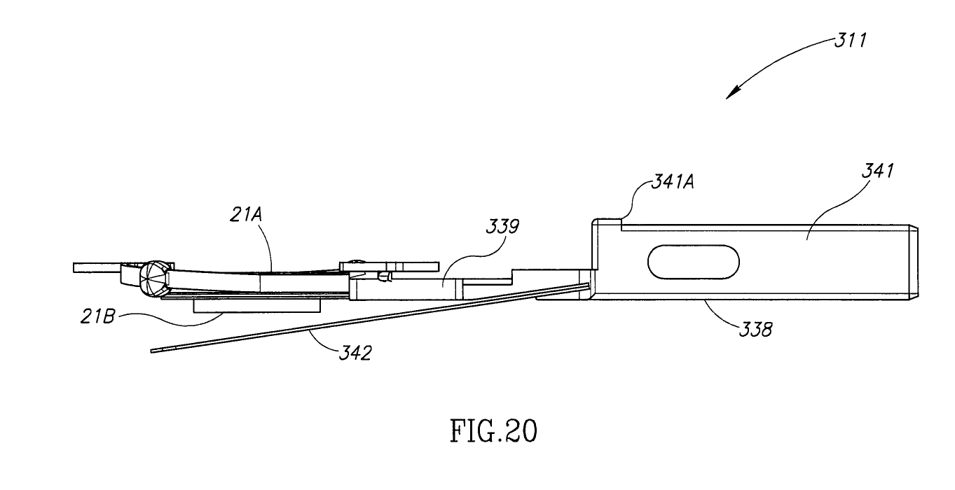

FIGS. 12 to 20 show injector apparatus 100B similar in construction and operation as injector apparatus 100A. The injector apparatus 100B includes a syringe-like injector 200B for use with an interchangeable cartridge 300 fitted with a pre-loaded IOL 10.

FIGS. 12 to 15 show the syringe-like injector 200B is similar to the syringe-like injector 200A and differs therefrom insofar the former 200B does not include the compression member tracks 214 with their respective left indentations 222 and right indentations 223, the pair of sidewall cutouts 219, the pair of overhang recesses 221, and the central lens support recess 224. The syringe-like injector 200B includes a central longitudinal directed cartridge track 272, left and right elongated slots 273A and 273B and left and right inwardly directed protrusions 274A and 274B correspondingly formed in the left and right side walls 213A and 213B between their trailing wall sections 216A and 216B and their leading wall sections 217A and 217B.

The syringe-like injector 200B includes a hand operated injector plunger 276 which is also manually urged from an initial outward set-up position to a final inward injection position for injecting the intraocular lens 10. The injector plunger 276 differs from the injector plunger 206 insofar that the former 276 has a leading end 277 for snap fit engagement with a driven plunger of the cartridge 300. Also the injector plunger 276 has a shaft 278 formed with a longitudinal groove 279 for engaging a downwards directed key 281 formed on an underside of the upper handgrip component 241 for preventing the injector plunger 276 from inadvertently sliding out from the syringe-like injector 200B on backwards tilting of the same.

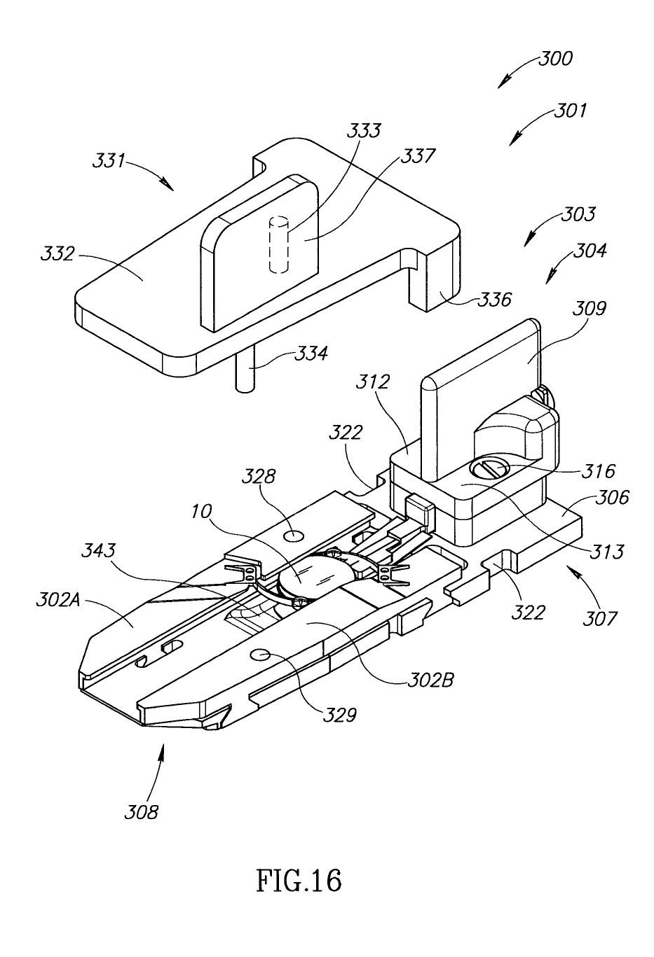

FIGS. 16 to 20 show the cartridge 300 includes an intraocular lens support arrangement 301 having left and right longitudinal directed elongated compression members 302A and 302B for initially supporting the IOL 10 in an unstressed state with topside and underside optical surfaces 21A and 21B, a first safety latch arrangement 303 for preventing inadvertent IOL compression and a second safety latch arrangement 304 for preventing inadvertent manual urging of the injector plunger 276 to its final inward injection position.

The cartridge 300 includes a generally rectangular cartridge base 306 having an upper surface 306A, a lower surface 306B, a trailing handheld cartridge end 307 and a leading delivery cartridge end 308. The cartridge 300 includes a handheld cartridge grip 309 mounted on the cartridge base 306 at its trailing handheld cartridge end 307 for enabling manipulation of the cartridge 300 and storing a driven plunger 311 for injecting the IOL 10. The cartridge grip 309 has a two part construction and includes an upper member 312 mounted on a support member 313. The support member 313 is formed with a longitudinal passage 314 for slidingly supporting the driven plunger 311. Assembly screws 316 mount the cartridge grip 309 on the trailing handheld cartridge end 307. Alternative assembly techniques can be employed for assembling the cartridge grip 309 including snap fit, gluing, and the like.

The cartridge base 306 includes a left pair of transverse directed grooves 317 including a left leading groove 317A, a left trailing groove 317B, and a right pair of transverse directed grooves 318 including a right leading groove 318A, and a right trailing groove 318B. The cartridge base 306 further includes a trailing left throughgoing bore 319 and a leading right throughgoing bore 321. The cartridge base 306 is formed with a pair of opposite cutouts 322 on opposite sides of the handheld cartridge grip 309 for use in correct alignment with the syringe-like injector 200B.

The compression members 302 are slidingly mounted on the cartridge base 306 from an initial locked position to a final compression position for applying the opposite compression forces for squeezing the IOL 10 from an unstressed generally circular shape to a stressed elongated shape. The compression members 302 are recessed with respect to the cartridge base 306 in their final compression position.

The compression members 302A and 302B have upper surfaces 323A and 323B and lower surfaces 324A and 324B. The upper surface 323A supports the left attachment plate 17A and the upper surface 323B supports the right attachment plate 17B in the set-up position of the cartridge 300. The lower surface 324A includes a pair of feet 326 for sliding sideways along the left pair of grooves 317. The lower surface 324B includes a pair of feet 327 for sliding sideways along the right pair of grooves 318. The left compression member 302A includes a throughgoing bore 328 in registration with the left throughgoing bore 319 in its initial locking position. The right compression member 302B includes a throughgoing bore 329 in registration with the right throughgoing bore 321 in its initial locking position.

The safety latch arrangement 303 includes a cartridge locking member 331 for locking the compression members 302 in their initial locked position to avoid inadvertent squeezing of the IOL 10. The cartridge locking member 331 includes a transparent crosspiece 332 for enable viewing of the underlying IOL 10, a left downward directed pin 333 for insertion through the throughgoing bore 328 into the throughgoing bore 319 for locking the left compression member 302A, and a right downward directed pin 334 for insertion through the throughgoing bore 329 into the throughgoing bore 321 for locking the right compression member 302B. The pins 333 and 334 protrude beyond the cartridge's lower surface 306B such that the cartridge locking member 331 is urged upwards on mounting the cartridge 300 into the injector 200B to distance it from the cartridge base 306 to assist a user to remove same. Also, raising the cartridge locking member 331 with respect to the cartridge base 306 ensures the topside optical surface 21A will not be scratched or be otherwise damaged on forward displacement of the IOL 10 relative to the compression members 302. The cartridge locking member 331 also includes a pair of trailing stops 336 for stopping forward displacement of the cartridge 300 along the syringe-like injector 200B and a handgrip 337 for assisting a user to upwardly remove same.

The driven plunger 311 has a shaft 338 with a leading driven plunger end 339 and a trailing driven plunger end 341. The leading driven plunger end 339 forms part of the intraocular lens support arrangement 301 for supporting the haptics main body 14 while its left and right attachment plates 17A and 17B rest on the compression members 302 in a non-flexed state to avoid mechanical strains. The trailing driven plunger end 341 is initially deployed inside the handheld cartridge grip 309 and is designed for snap fit connection with the injector plunger 276. The trailing driven plunger end 341 has a leading edge 341A for stopping against the upper cartridge grip member's leading edge 312A for preventing the driven plunger 311 from inadvertently sliding out from the cartridge 300 on backwards tilting of same.

The driven plunger 311 also includes a lens support 342 which is initially downwardly disposed in a central longitudinally directed lens support recess 343 formed midway along the top surface 306A. The lens support recess 343 includes an inclined ramp 344 towards the leading cartridge delivery end 308 for upwardly urging the lens support 342 against the IOL's underside optical surface 21B on forward displacement of the driven plunger 311 by the injector plunger 276.

The cartridge 300 includes an upright injector plunger stopper 346 with a pair of opposite downward directed keys 347A and 347B for insertion into key slots 348A and 348B lateral of the longitudinal passage 314 for stopping forward displacement of the driven plunger 311 for enabling the injector plunger 276 to snap fit engage same.

The handheld cartridge grip 309 has a trailing surface 309A fitted with a downward depending snap fit member 349 for snap fitting into a transverse directed slit 351 formed on the injector plunger 276 when the injector plunger 276 has been urged sufficiently far forward that the IOL 10 is in line with the leading edges of the compression members 302.

Operation of Injector Apparatus with Interchangeable Cartridge

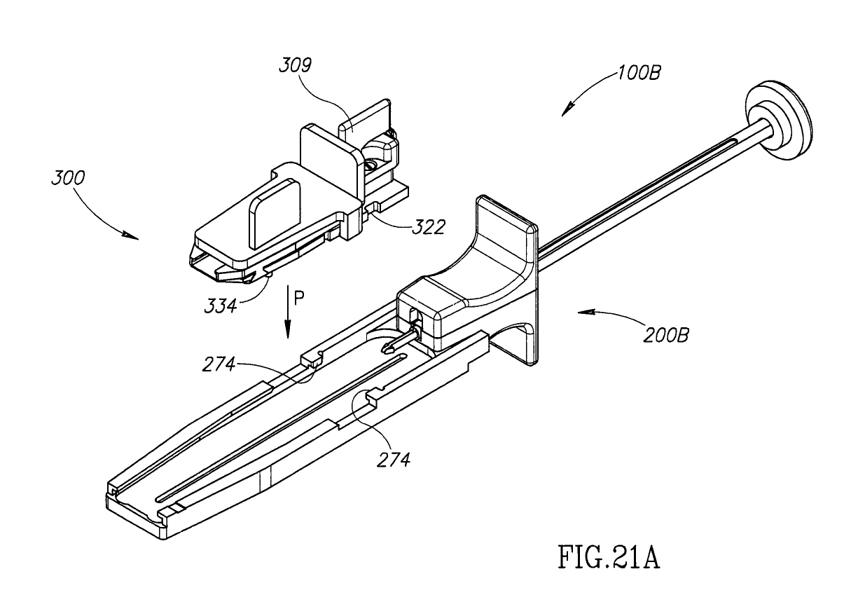

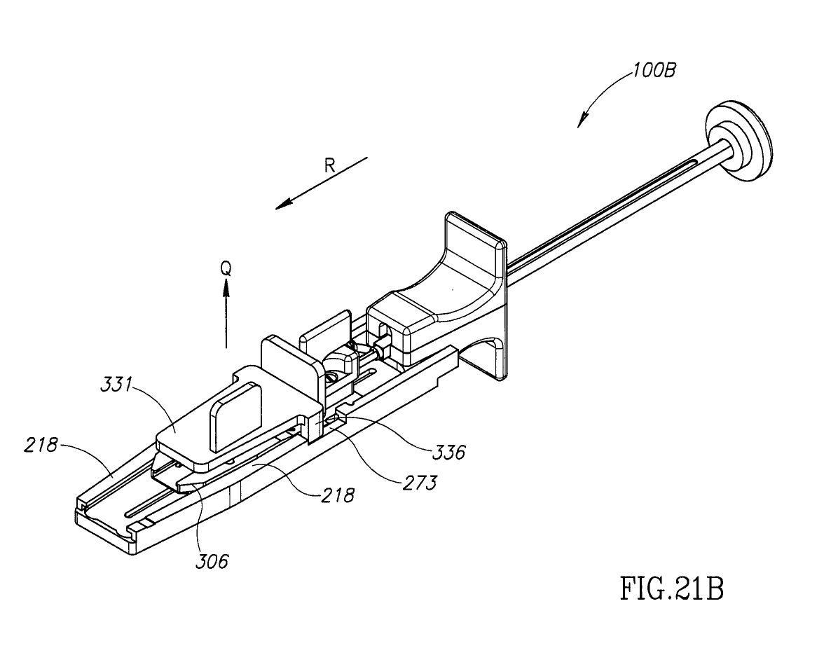

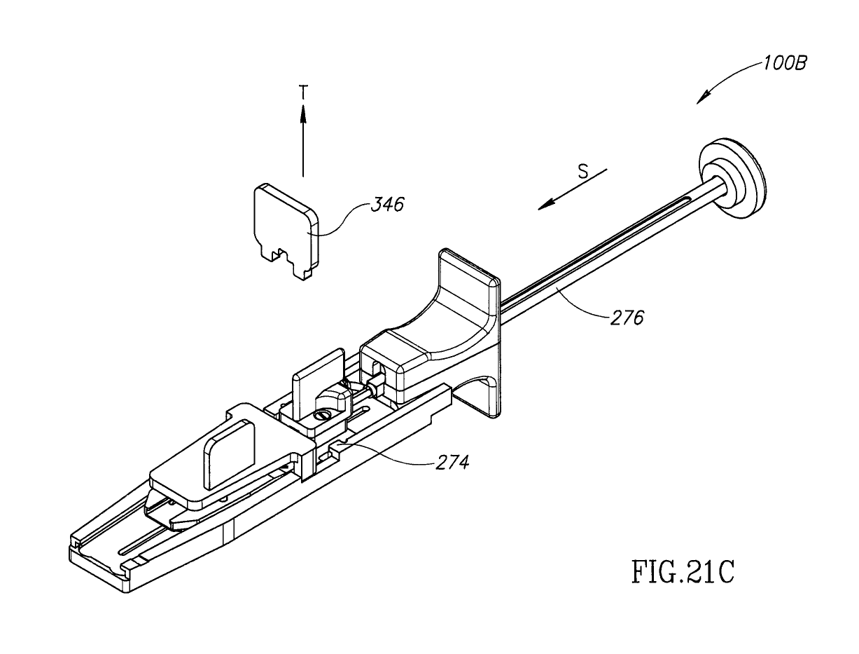

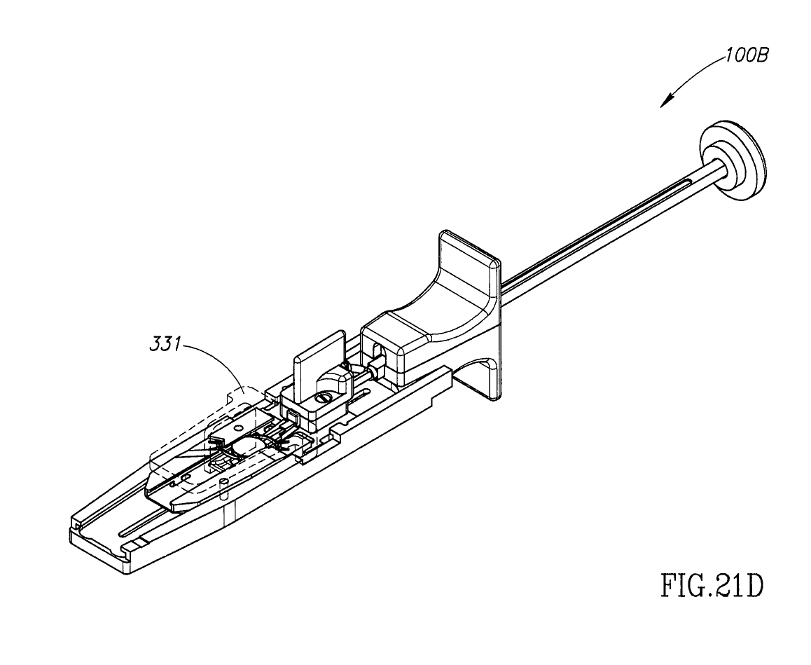

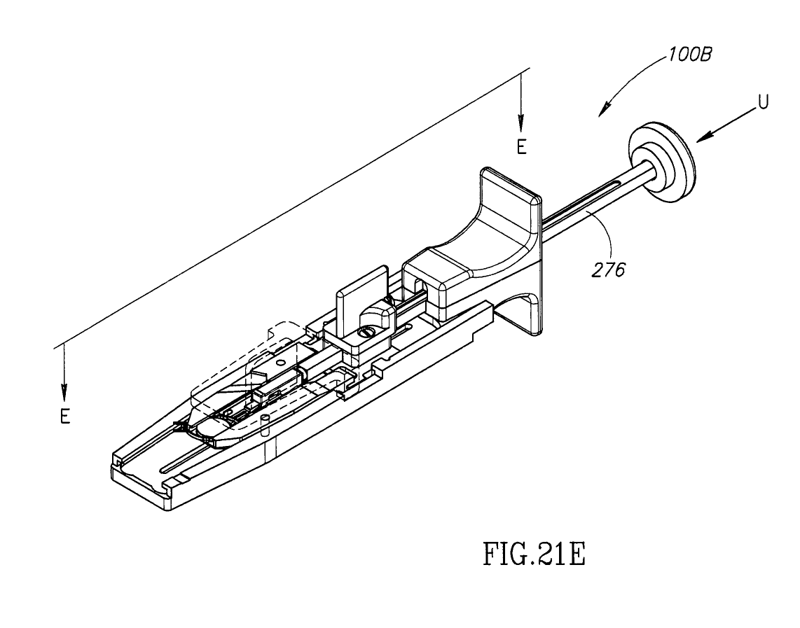

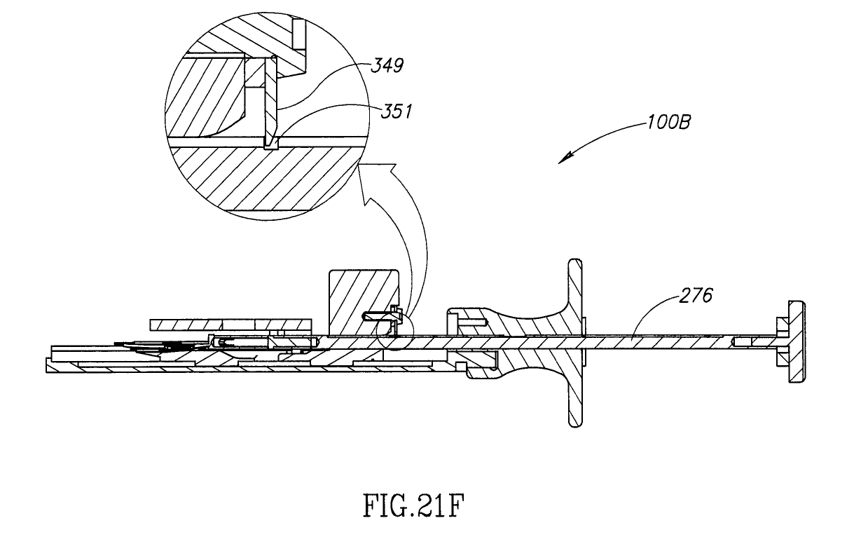

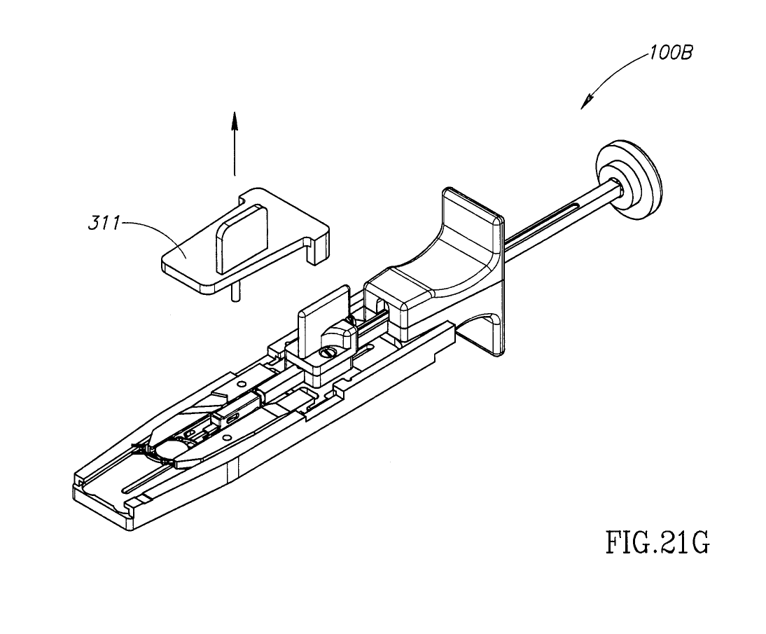

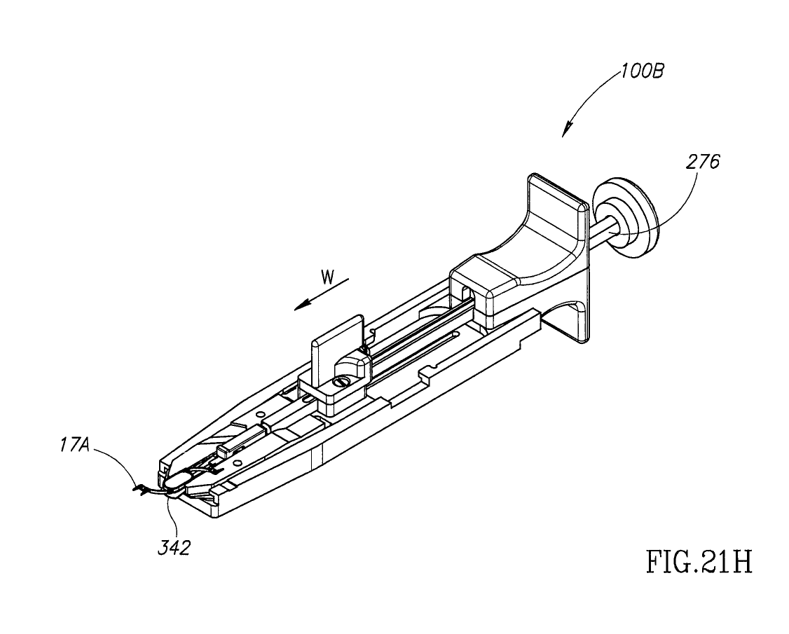

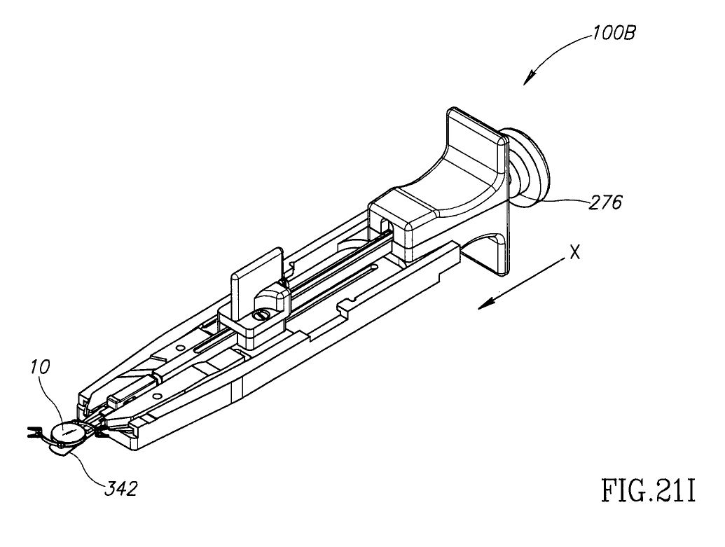

FIGS. 21A to 21I show operation of the injector apparatus 100B as follows:

FIG. 21A shows a user gripping the handheld cartridge grip 309 for inserting the cartridge 300 with the pre-installed IOL 10 into the syringe-like injector 200B in its initial loading position as denoted by arrow P. The cartridge 300 is correctly aligned with the syringe-like injector 200B on registration of the opposite cutouts 322 with the inwardly directed protrusions 274. The insertion urges the cartridge locking member 331 upwards relative to the cartridge base 306 as denoted by arrow Q (see FIG. 21B).

FIG. 21B shows the user gripping the handheld cartridge grip 309 for pushing the cartridge 300 forward from its initial loading position as denoted by arrow R until the cartridge 300 stops against the syringe-like injector 200B on abutment of the trailing stops 336 at the leading ends of the elongated slots 273 (see FIG. 21C). The cartridge base 306 slides under the inwardly directed protrusions 274.

FIG. 21C shows the user urging the injector plunger 276 forward as denoted by arrow S to snap fit with the driven plunger 311 and the user upwardly detaching the injector plunger stopper 346 as denoted by arrow T such that the driven plunger 311 is free to be pushed forward relative to the cartridge base 306.

FIG. 21D shows the injector assembly 100B in the same operative state as in FIG. 21C with the cartridge locking member 331 shown transparent to show the IOL 10 in its initial non-stressed state in the cartridge 300.

FIGS. 21E and 21F shows the user depressing the injector plunger 276 forward as denoted by arrow U to urge the driven plunger 311 forward relative to the compression members 302. Forward displacement of the drive plunger 311 urges the lens support 342 along its ramp 344 to bear against the underside optical surface 21B of the IOL 10 which still remains in its original non-stressed state. The user continues to urge the injector plunger 276 forward until he hears the snap fit member 349 click into the transverse directed slit 351 to indicate the IOL 10 has reached the leading edges of the compression members 302. The IOL 10 remains in its unstressed generally circular shape supported underneath by the lens support 342.

FIG. 21G shows the user upwardly detaching the cartridge locking member 331 as denoted by arrow V to release the compression members 302 relative to the cartridge base 306.

FIG. 21H shows the user gripping the handheld cartridge grip 309 and pushing the cartridge 300 forward towards the leading delivery end 204 as denoted by arrow W until the cartridge 300 stops at the end of the cartridge track 272. The side wall overhangs 218 simultaneously urge the compression members 302 to slide one towards other during forward displacement of the cartridge base 306. The compression members 302 progressively squeeze the initially generally circular IOL into its stressed elongated shape. The opposite compression forces cause the topside optical surface 21A to bulge upwards while the lens support 342 prevents the underside optical surface 21B bulging downwards. The elongation of the IOL causes the left attachment plate 17A to extend forward on the left compression member 302A's upper surface 323A. Similarly, the elongation of the IOL causes the right attachment plate 17B to slightly extend rearward on the right compression member 302B's upper surface 323B.

The user inserts the leading left attachment plate 17A and the leading tip of lens support 342 through a corneal incision prepared in a subject's cornea and then presses the leading edges of the compression members 302 against the subject's cornea for partial insertion of the IOL 10 into the subject's eye. For the final step, the user manually urges the injector plunger 276 as denoted by arrow X to its final inward injection position for manually urging the lens support 342 for fully injecting the IOL 10 into the subject's eye (see FIG. 21I). The IOL 10 reverts to its original non-stressed shape within the subject's eye ready for implantation. The user withdraws the injector apparatus 200A and proceeds to implant the IOL as illustrated and described in commonly owned PCT International Application No. PCT/IL2007/001056 entitled Intraocular Lens Implantation Kit and published under PCT International Publication No. WO 2008/023379.

While the invention has been described with respect to a limited number of embodiments, it will be appreciated that many variations, modifications, and other applications of the invention can be made within the scope of the appended claims.

1. Injector apparatus for injecting an intraocular lens into a human eye, the intraocular lens having an optical axis and including an optical lens and at least two outward extending haptics for anchoring the intraocular lens in the human eye, the optical lens having a pair of opposite optical surfaces and being compressible on application of opposite compression forces in a plane perpendicular to its optical axis from an unstressed generally circular shape to a stressed elongated shape whereupon the optical lens outwardly bulges along its optical axis with respect to its unstressed generally circular shape, the injector apparatus comprising:
(a) a syringe-like injector having a longitudinal axis and including (i) an elongated injector base having a trailing handheld end and a leading delivery end, and (ii) a hand operated injector plunger slidingly mounted on said injector base for being manually urged from an initial outward set-up position to a final inward injection position for injecting the intraocular lens, said hand operated injector plunger including a thumb depressed plunger head and a shaft having a leading shaft end opposite said plunger head, said leading shaft end including a lens support for bearing against the underside optical surface at an intermediate position of the intraocular lens between said trailing handheld end and said leading delivery end;
(b) an intraocular lens support arrangement for initially supporting the intraocular lens in its unstressed state at said trailing handheld end such that its optical lens overlies said injector base with a topside optical surface of the pair of opposite optical surfaces facing away from said injector base and an underside optical surface of the pair of opposite optical surfaces facing toward said injector base; and
(c) a pair of compression members for sliding towards one another for applying opposite compression forces to the intraocular lens disposed therebetween in a plane perpendicular to its optical axis for compressing its optical lens from its initial unstressed state at said trailing handheld end into its stressed elongated shape at said leading delivery end,
the injector apparatus being shaped and dimensioned such that when said hand operated injector plunger is manually urged from said initial outward set-up position towards said final inward injection position, (1) initial displacement of said hand operated injector plunger urges said lens support to bear against the underside optical surface at said intermediate position, (2) further displacement of said hand operated injector plunger urges said pair of compression members to apply said opposite compression forces for compressing the intraocular lens into its stressed elongated shape with its topside optical surface bulging upwards away from said injector base and said lens support preventing its underside optical surface from bulging downwards towards said injector base, (3) still further displacement of said hand operated injector plunger urges said pair of compression members and said lens support together with the intraocular lens in its stressed elongated shape to said leading delivery end, and (4) yet still further displacement of said hand operated injector plunger to said final inward injection position urges said lens support together with the intraocular lens beyond said leading delivery end.

2. Apparatus according claim 1 and further comprising a first safety latch arrangement for stopping said relative movement of said pair of compression members one towards the other for preventing inadvertent compression of the intraocular lens.

3. Apparatus according to claim 1 and further comprising a second safety latch arrangement for preventing inadvertent manual urging of said injector plunger to said final inward injection position for injecting the intraocular lens.

4. Apparatus according to claim 1 wherein said syringe-like injector is integrally formed with said intraocular lens support arrangement pre-loaded with the intraocular lens having a topside optical surface facing away from and an underside optical surface facing towards said injector base, and said pair of compression members.

5. Apparatus according to claim 1 wherein an interchangeable cartridge including said intraocular lens support arrangement pre-loaded with an intraocular lens is manually loaded into said syringe-like injector prior to intended operation of the injector apparatus whereupon the intraocular lens has a topside optical surface facing away from and an underside optical surface facing towards said injector base.

6. Apparatus according to claim 5 wherein said interchangeable cartridge includes said pair of compression members for initially supporting the intraocular lens and thereafter applying the opposite compression forces.
